(19) United States
(12) Patent Application Publication
(10) Pub. No.: US 2023/0351754 A1
(43) Pub. Date: Nov. 2, 2023

(54) IMAGE-BASED ANALYSIS OF A TEST KIT

(71) Applicant: Exa Health, Inc., Los Altos, CA (US)

(72) Inventors: Siddarth Satish, Redwood City, CA (US); Steven Scherf, Oakland, CA (US); Charles Peterson Carroll, Berkeley, CA (US); Mayank Kumar, Sunnyvale, CA (US)

(21) Appl. No.: 17/282,482

(22) PCT Filed: Apr. 1, 2021
(86) PCT No.: PCT/US21/25359

§ 371 Date: Apr. 2, 2021

Related U.S. Application Data
(63) Continuation of application No. 63/004,431, filed on Apr. 2, 2020.

(51) Int. Cl. G06V 20/50; H04N 23/60; G06V 10/22; G16H 10/40; G16H 40/67

(52) U.S. Cl. CPC G06V 20/50; H04N 23/64; G06V 10/235; G16H 10/40; G16H 40/67

(57) ABSTRACT

A device may be configured to function as an image-based analyzer for one or more test kits. The device guides a user in capturing an image of a test kit within an appropriate window of time. The device analyzes the image of the test kit, recognizes one or more features of the test kit depicted in the image, obtains one or more results indicated by the one or more recognized features, and provides one or more of the one or more results. The device may be configured to provide some or all of the results. The results may be displayed visually on a display screen of the device, presented audibly via speaker of the device, or both.

### RELATED APPLICATION

This application is a national phase entry of PCT/US21/25359, titled “IMAGE-BASED ANALYSIS OF A TEST KIT,” and filed Apr. 1, 2021, which claims the priority benefit of U.S. Provisional Patent Application No. 63/004,431, titled “IMAGE-BASED ANALYSIS OF A TEST KIT,” and filed Apr. 2, 2020, which is incorporated herein by reference in its entirety.

### TECHNICAL FIELD

The subject matter disclosed herein generally relates to the technical field of special-purpose machines that facilitate healthcare testing, including software-configured computerized variants of such special-purpose machines and improvements to such variants, and to the technologies by which such special-purpose machines become improved compared to other special-purpose machines that facilitate healthcare testing. Specifically, the present disclosure addresses systems and methods to facilitate image-based analysis of a test kit.

### BACKGROUND

A device may be configured (e.g., by suitable software, such as an app) to capture an image using a camera of the device. The device may thereafter communicate the captured image to another device or other machine via a network.

### BRIEF DESCRIPTION OF THE DRAWINGS

Some embodiments are illustrated by way of example and not limitation in the figures of the accompanying drawings.

FIG. 1 is a diagram illustrating example screens of a mobile app that enables a user to perform a healthcare test on himself or herself and guides the user through image-based analysis of a test kit within a specified window of time, according to some example embodiments.

FIG. 2 is an annotated table describing example messages that the mobile app may cause to be presented to the user, based on results of the image-based analysis of the test kit, according to some example embodiments.

FIG. 3 is photograph of several test kits, illustrating example features suitable for image-based analysis, according to some example embodiments.

FIG. 4 is a flowchart illustrating operations of a device (e.g., as configured by the mobile app) in performing a method for image-based analysis of a test kit, according to some example embodiments.

FIG. 5 is a block diagram illustrating components of a machine, according to some example embodiments, able to read instructions from a machine-readable medium and perform any one or more of the methodologies discussed herein.

### DETAILED DESCRIPTION

Example methods (e.g., algorithms) facilitate image-based analysis of one or more test kits, and example systems (e.g., special-purpose machines configured by special-purpose software) are configured to facilitate image-based analysis of one or more test kits. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will be evident to one skilled in the art, however, that the present subject matter may be practiced without these specific details.

Healthcare testing is often performed by a healthcare worker on a patient, such that results can be reported by the healthcare worker both to the patient and to a central authority, such as government health department. The central authority may have a rule that disallows patients from performing healthcare testing on themselves, out of concerns that patients might not analyze their test kits within the proper window of time, that patients might not reliably interpret results of their test kits, that patients might not report the results of their healthcare tests to the central authority, or any combination of these concerns.

A device (e.g., a mobile device, such as a smartphone) may be configured (e.g., by a mobile app or other suitable software, hardware, or both) to function as an image-based analyzer for one or more test kits. A user may use a test kit to perform a healthcare test. For example, the test kit may be configured to administer or otherwise perform a polymerase chain reaction (PCR) test for presence of a virus, an antibody test for presence of antibodies for that virus, or a combined test for both. The discussion herein also contemplates one or more of various other healthcare tests, such as human immunodeficiency virus (HIV) tests (e.g., viral load tests, antibody tests, and combination tests), hepatitis tests, pregnancy tests, or other tests that can be performed on a suitable sample (e.g., of a body fluid, such as blood, saliva, or urine) using an appropriate test kit (e.g., a lateral flow assay (LFA) test kit). For example, the device may download, install, and execute a mobile app specifically configured for image-based analysis of a test kit, and the mobile app may cause the device to perform any one or more of the operations discussed herein.

As configured (e.g., by the mobile app), the device guides the user in capturing an image of the test kit within an appropriate window of time. For example, the device may instruct the user to capture the image after a predetermined minimum wait time has elapsed after using the test kit, provide a timer (e.g., a countdown timer, with visible prompts, audible prompts, or both), warn the user if the user attempts to capture the image too soon, prompt the user to capture the image after the predetermined minimum wait time has elapsed and before a predetermined maximum wait time has elapsed, warn the user if the predetermined maximum wait time is drawing near (e.g., within a threshold warning period), notify the user that the predetermined maximum wait time has expired, disallow the user from proceeding if the predetermined maximum wait time has expired without capture of an image of the test kit, or any suitable combination thereof.

If the image of the test kit has been captured (e.g., within the appropriate period of time), the device analyzes the image of the test kit, recognizes one or more features of the test kit depicted in the image, obtains (e.g., generates) one or more results indicated by the one or more recognized features, and provides one or more of the one or more results. For example, a test kit may take the form of a test strip for detecting the presence of antibodies for a particular virus (e.g., SARS-CoV-2, the virus that causes COVID-19 disease). As configured (e.g., by the mobile app), the device analyzes an image of a test strip and recognizes (e.g., using computer vision or other artificial intelligence for optical recognition) one or more indicators visible in the image of the test strip. Examples of such indicators include a result window and markings adjacent or otherwise proximate thereto (e.g., “C” for “control,” “M” for short-term immunoglobulin M (IgM) antibodies, and “G” for long-term immunoglobulin G (IgG) antibodies). The device may additionally recognize the shape of the test strip, a sample insertion aperture (e.g., a blood droplet input hole), a name of a manufacturer of the test kit, a name of the test kit, a model number of the test kit, or any suitable combination thereof, any one or more of which may be factors used by the device to recognize the result window and its corresponding markings.

With the result window recognized, the device identifies or otherwise obtains the results themselves by recognizing presence or absence of marks (e.g., bars, squares, or dots), lengths of gradients, presence or absence of colors (e.g., blue versus pink), or any suitable combination thereof, within the result window and at locations corresponding (e.g., by virtue of close proximity) to the recognized markings. A test kit for detecting the presence or absence of antibodies for the SARS-CoV-2 virus may have the markings “C,” “M,” and “G” near its result window, and the device may recognize the presence or absence of a respective mark (e.g., a bar) for each of the markings. For example, a bar next to the “C” marking may be recognized as presence of a control within the test kit, and the device may check for this recognition first before checking the other markings to ascertain that the test kit is functioning normally and ready for interpretation. As another example, recognition of impossible or nonsensical results may indicate a spoiled test kit, and the device may respond by switching to an error mode, presenting an error alert, or otherwise treating the test kit as unusable for obtaining accurate results.

According to various example embodiments, the device is configured to provide some or all of the obtained results. The results may be displayed visually on a display screen of the device, presented audibly via speaker of the device, or both. The results may be sent to another device, such as another device of the user, a device of a healthcare worker (e.g., a doctor or a nurse), a device or other machine (e.g., a server machine) of a hospital or government office, or any suitable combination thereof. The device may provide results within a predetermined window of time (e.g., a validity period for the test kit or for the type of test kit), and the device may perform error checking to ensure compliance with such a predetermined window of time. In some example embodiments, the device is configured to send one or more results first to a predetermined server machine (e.g., corresponding to a government office or other authoritative entity) before providing any results to the user, to ensure that the one or more results are reported.

In certain example embodiments, additional data is accessed and processed with one or more results from the test kit, to provide further results. For example, if a PCR test had previously been performed and its result is accessible (e.g., locally or via a network) by the device, the device may access the PCR result, generate a further result based on the PCR result and one or more of the test kit results, and provide the further result (e.g., to the user, to an authoritative entity, or to both). Where the test kit detects presence or absence of antibodies for the SARS-CoV-2 virus, the device may access a PCR result for presence or absence of the SARS-CoV-2 virus, generate an assessment of the user's readiness to go to work based on the PCR result and the test kit results, and then provide the assessment (e.g., a readiness score) as described above.

In various example embodiments, the device is configured to perform one or more of the methodologies discussed herein using any one or more of various computer vision techniques, including those utilizing machine-learning (e.g., deep learning) to analyze the image captured by the device and recognize the test kit or any portion thereof. For example, using such techniques, various example embodiments of the device may recognize individual marks (e.g., individual bars) or patterns of multiple marks (e.g., configurations of multiple bars). In the case of recognizing patterns of multiple marks, each possible pattern may be classified by a trained classifier (e.g., trained by deep learning) for use in classifying actual patterns of multiple marks in images captured by the device.

In some example embodiments, the device is configured to generate a prediction for a health status of the user (e.g., whether the user is currently infected with a virus, was previously infected with the virus, is currently immune to the virus, or any suitable combination thereof). Such a prediction may be generated and provided (e.g., communicated, presented, or both) by the device based on the identified results of the test kit, one or more accessed results of another test (e.g., a PCR test), one or more symptoms indicated by the user as being experienced by the user, or any suitable combination thereof.

According to various example embodiments, the device provides one or more results in any one or more of the following forms: binary (e.g., yes or no, present or absence, infected or uninfected, etc.), marks present (e.g., bars showing) in the result window of the test kit, marks absent (e.g., bars missing) from the result window of the test kit, a bounding box drawn around one or more of the marks in the result window, or any suitable combination thereof.

According to certain example embodiments, the device provides further information generated, determined, or otherwise obtained based on the identified results of the test kit. Examples of such further information include: a recommendation on what the user should do next regarding the results, a risk level for contracting a disease (e.g., generated or otherwise obtained based on the results of the test kit), a clearance notification that indicate absence of disease (e.g., generated or otherwise obtained based on the results of the test kit), or any suitable combination thereof.

According to some example embodiments, when a test kit is not recognized by the device, the device presents the user with a graphical user interface by which the user can manually define (e.g., by drawing bounding box) an indicator on the test kit, the result window on the test kit, one or more markings for the result window, one or more marks in the result window, or any suitable combination thereof. The device accordingly may be configured to modify its image analysis operation, its result identification operation, or both, based on such user-defined features of the test kit. Alternatively, the device may be configured to upload definition data for such user-defined features to a server machine configured to perform such modifications thereon and reply with an app update that reconfigures the device to perform the modified operations. The server machine may also provide the app update to one or more other devices to modify similar operations performed thereon.

In various example embodiments, some or all of the functionality described above for the mobile app is also available via a web interface hosted by a web server. Accordingly, the systems and methods discussed herein may be flexibly deployed in the user's environment (e.g., at home or at work), as well as in various healthcare settings, such that doctor visits, urgent care, and emergency room treatment can benefit from image-based analysis of one or more test kits used by the user.

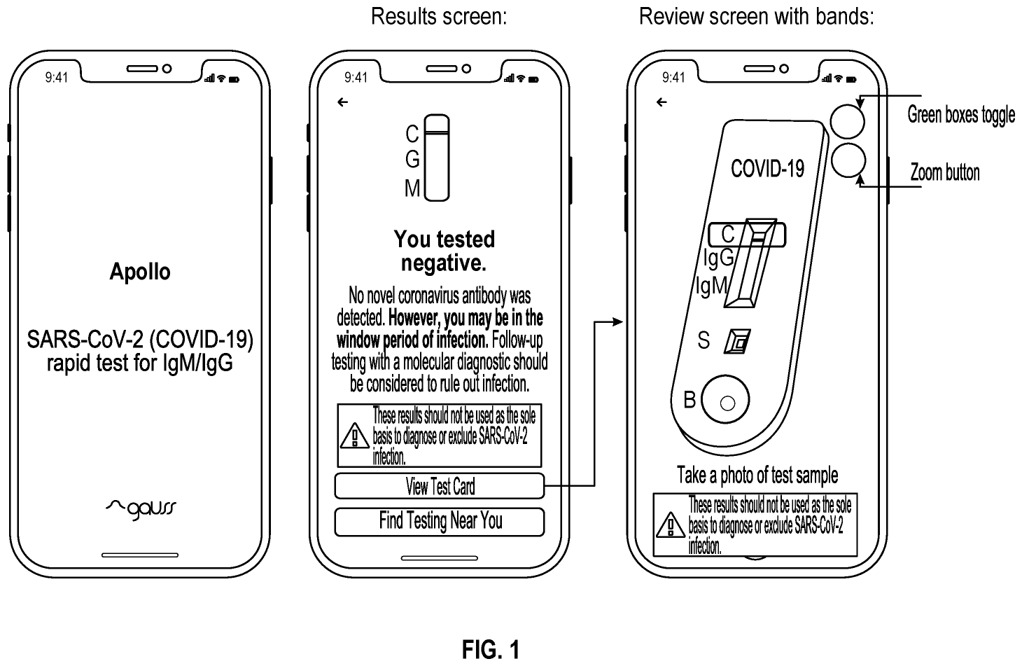

FIG. 1 is a diagram illustrating example screens of a mobile app that enables a user to perform a healthcare test on himself or herself and guides the user through image-based analysis of a test kit within a specified window of time, according to some example embodiments. Any one or more of the above-described operations may be performed by a device configured by the mobile app illustrated in FIG. 1

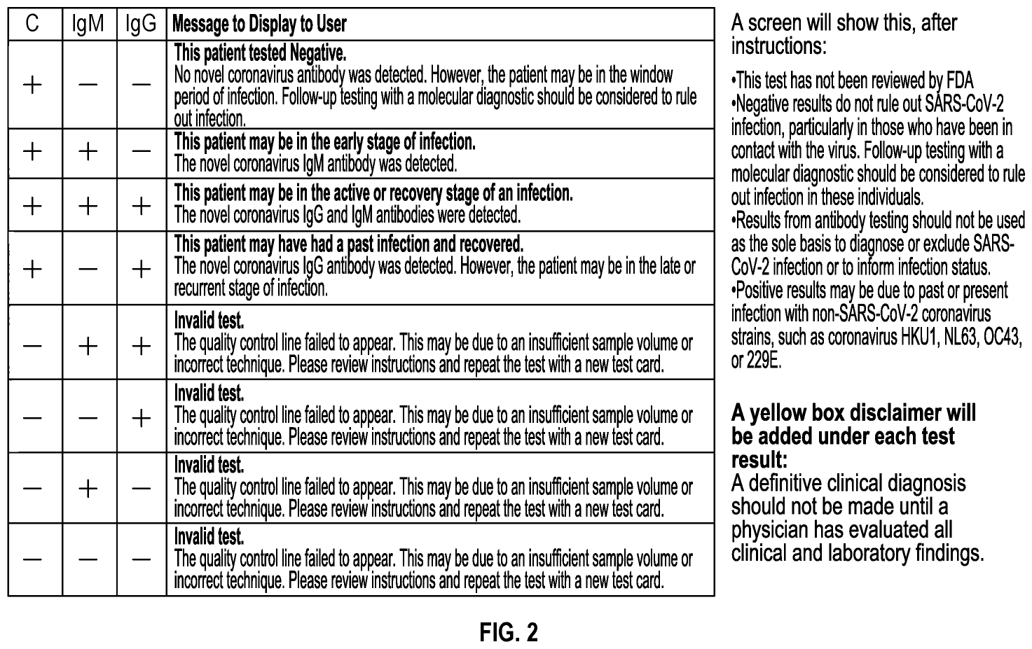

FIG. 2 is an annotated table describing example messages that the mobile app may cause to be presented to the user, based on results of the image-based analysis of the test kit, according to some example embodiments. Each of the example messages shown in FIG. 2 indicate a different result recognizable from one or more marks displayed in the result window of a test kit.

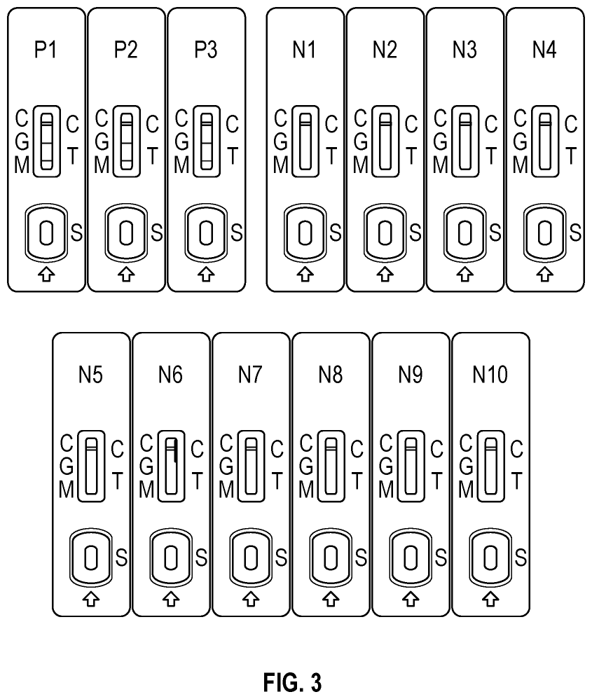

FIG. 3 is photograph of several test kits, illustrating example features suitable for image-based analysis, according to some example embodiments. The test kits shown each include a result window with adjacent markings (e.g., “C,” “G,” and “M”), a sample insertion aperture, and at least one mark (e.g., a bar) visible though the result window.

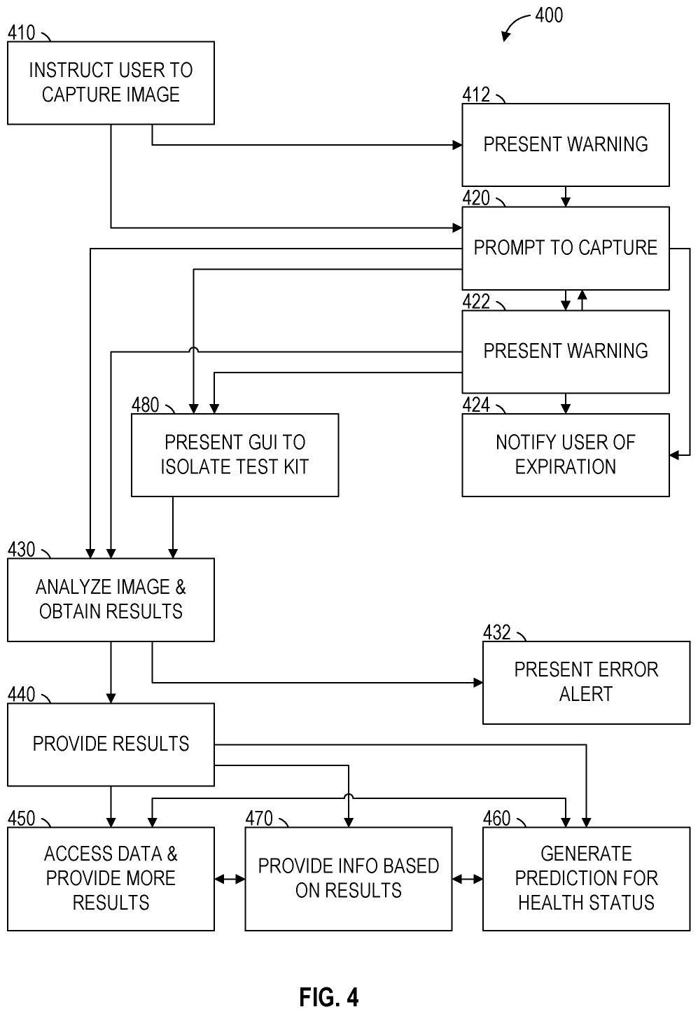

FIG. 4 is a flowchart illustrating operations of a device (e.g., as configured by a mobile app) in performing a method 400 for image-based analysis of a test kit, according to some example embodiments. Operations in the method 400 may be performed by any device (e.g., a mobile device, such as a smartphone, tablet computer, or a smartwatch), using one or more processors (e.g., microprocessors or other hardware processors), or using any suitable combination thereof. As shown in FIG. 4, the method 400 includes one or more of operations 410, 412, 420, 422, 424, 430, 432, 440, 450, 460, 470, and 480.

In operation 410, the device instructs its user to capture an image after a predetermined minimum wait time has elapsed after using the test kit. That is, the device instructs the user to wait until the predetermined minimum wait time has elapsed, and then capture the image of the test kit. The device may accordingly display or otherwise provide a timer (e.g., a countdown timer, with visible prompts, audible prompts, or both). Depending on the test kit, the predetermined minimum wait time may be anywhere from a few seconds (e.g., 3, 4, 5, 8, or 10 seconds), to several seconds (e.g., 12, 15, 30, or 45 seconds), to a few minutes (e.g., 1, 1.5, 2, 2.5, 3, 4, 5, 8, or 10 minutes), to several minutes (e.g., 12, 15, 30, or 45 minutes).

If the user attempts to capture the image too soon (e.g., as detected by the device), in operation 412, the device displays or otherwise presents (e.g., audibly or haptically) a warning that the user is attempting to capture the image too soon.

After expiration of the predetermined minimum wait time (e.g., as detected by the device), and prior to expiration of a predetermined maximum wait time, in operation 420, the device prompts (e.g., visually, audibly, haptically, or any suitable combination thereof) the user to capture the image of the test kit.

If the user has not yet captured the image (e.g., as detected by the device), and the predetermined maximum wait time is drawing near (e.g., within a predetermined threshold warning period, as determined by the device), in operation 422, the device warns (e.g., visually, audibly, haptically, or any suitable combination thereof) the user that the predetermined maximum wait time is drawing near (e.g., within the predetermined threshold warning period). Depending on the test kit, the predetermined maximum wait time may be anywhere from a few seconds (e.g., 5, 8, or 10 seconds), to several seconds (e.g., 12, 15, 30, or 45 seconds), to a few minutes (e.g., 1, 1.5, 2, 2.5, 3, 4, 5, 8, or 10 minutes), to several minutes (e.g., 12, 15, 30, or 45 minutes), to a few hours (e.g., 1, 1.5, 2, 2.5, or 3 hours), so long as the predetermined maximum wait time is greater than the predetermined minimum wait time.

If the user has not yet captured the image (e.g., as detected by the device), and the predetermined maximum wait time has expired (e.g., as determined by the device), in operation 424, the device notifies (e.g., visually, audibly, haptically, or any suitable combination thereof) the user that the predetermined maximum wait time has expired. Such a notification may additionally or alternatively inform the user that the test kit is no longer valid (e.g., spoiled or otherwise unreliable), that a new test kit should be used, or both. In some example embodiments, the device (e.g., as configured by the mobile app) disallows the user from proceeding further to subsequent screens of the mobile app after the predetermined maximum wait time has expired without capture of an image of the test kit.

If the image of the test kit has been captured (e.g., within the appropriate period of time, as detected by the device), in operation 430, the device analyzes the image of the test kit, recognizes one or more features of the test kit depicted in the image, obtains (e.g., generates or determines) one or more results indicated by the one or more recognized features, or any suitable combination thereof. For example, a test kit may be or include a test strip for detecting the presence of antibodies for a particular virus (e.g., SARS-CoV-2, the virus that causes COVID-19 disease). In operation 430, the device may analyze an image of the test strip and recognize (e.g., using computer vision or other artificial intelligence for optical recognition) one or more indicators visible in the image of the test strip. As noted above, examples of such indicators include: a result window, one or more markings adjacent or otherwise proximate thereto (e.g., “C” for “control,” “M” for short-term immunoglobulin M (IgM) antibodies, and “G” for long-term immunoglobulin G (IgG) antibodies), or any suitable combination thereof. In operation 430, the device may additionally recognize the shape of the test strip, a sample insertion aperture (e.g., a blood droplet input hole), a name of a manufacturer of the test kit, a name of the test kit, a model number of the test kit, or any suitable combination thereof, and any one or more of these factors may be used by the device to recognize the result window and its one or more corresponding markings.

In various example embodiments, the device is configured to perform operation 430 using any one or more of various computer vision techniques, including those utilizing machine-learning (e.g., deep learning) to analyze the image captured by the device and recognize the test kit or any portion thereof. For example, using such techniques, various example embodiments of the device may recognize one or more individual marks (e.g., individual bars) or one or more patterns of multiple marks (e.g., configurations of multiple bars). In the case of recognizing patterns of multiple marks, each possible pattern may be classified by a trained classifier (e.g., trained by deep learning) for use in classifying actual patterns of multiple marks in images captured by the device.

As part of operation 430, the device identifies, generates, determines, interprets, or otherwise obtains the results by recognizing the presence or absence of one or more marks (e.g., bars, squares, or dots), the lengths of one or more gradients, the presence or absence of one or more colors (e.g., blue versus pink), or any suitable combination thereof, within the result window and at locations corresponding (e.g., by virtue of close proximity) to the recognized markings.

According to various example embodiments, the device provides one or more results in any one or more of the following forms: binary (e.g., yes or no, present or absent, infected or uninfected, etc.), marks present (e.g., bars showing) in the result window of the test kit, marks absent (e.g., bars missing) from the result window of the test kit, a bounding box drawn around one or more of the marks in the result window, or any suitable combination thereof.

According to various example embodiments, the device may determine one or more results that are impossible or nonsensical, which may indicate a spoiled test kit, and in operation 432, the device may accordingly respond by switching to an error mode, presenting an error alert, or otherwise treating the test kit as unusable for obtaining accurate results

In operation 440, the device provides one or more of the results obtained (e.g., generated) in operation 430. For example, one or more of the results may be displayed visually on a display screen of the device, presented audibly via speaker of the device, or both. One or more of the results may be sent to another device, such as another device of the user, a device of a healthcare worker (e.g., a doctor or a nurse), a device or other machine (e.g., a server machine) of a hospital or government office, or any suitable combination thereof. The device may provide one or more of the results within a predetermined window of time (e.g., a validity period for the test kit or for the type of test kit), and the device may perform error checking to ensure compliance with such a predetermined window of time. In some example embodiments, the device is configured to send one or more results first to a predetermined server machine (e.g., corresponding to a government office or other authoritative entity) before providing any results to the user, to ensure that the one or more results are reported.

In certain example embodiments, in operation 450, the device accesses additional data, processes the additional data with one or more results from the test kit, and provides (e.g., generates) one or more further results. For example, if a PCR test had previously been performed and its result is accessible (e.g., locally or via a network) by the device, the device may access the PCR result, generate a further result based on the PCR result and one or more of the test kit results, and provide the further result (e.g., to the user, to an authoritative entity, or to both). Where the test kit detects the presence or absence of antibodies for the SARS-CoV-2 virus, the device may access a PCR result for presence or absence of the SARS-CoV-2 virus, generate an assessment of the user's readiness to go to work based on the PCR result and the test kit results, and then provide the assessment (e.g., a readiness score) as described above.

In some example embodiments, in operation 460, the device generates a prediction for a health status of the user (e.g., whether the user is currently infected with a virus, was previously infected with the virus, is currently immune to the virus, or any suitable combination thereof). Such a prediction may be generated and provided (e.g., communicated, presented, or both) by the device based on the identified results of the test kit, one or more accessed results of another test (e.g., a PCR test), one or more symptoms indicated by the user as being experienced by the user, or any suitable combination thereof.

According to certain example embodiments, in operation 470, the device provides further information generated, determined, or otherwise obtained based on the identified results of the test kit. Examples of such further information include: a recommendation on what the user should do next regarding the results, a risk level for contracting a disease (e.g., generated or otherwise obtained based on the results of the test kit), a clearance notification that indicate absence of disease (e.g., generated or otherwise obtained based on the results of the test kit), or any suitable combination thereof.

According to some example embodiments, when a test kit is not recognized by the device, in operation 480, the device presents the user with a graphical user interface by which the user can manually define (e.g., by drawing bounding box) an indicator on the test kit, the result window on the test kit, one or more markings for the result window, one or more marks in the result window, or any suitable combination thereof. The device accordingly may be configured to modify its image analysis operation, its result identification operation, or both, based on such user-defined features of the test kit. Alternatively, the device may be configured to upload definition data for such user-defined features to a server machine configured to perform such modifications thereon and reply with an app update that reconfigures the device to perform the modified operations. The server machine may also provide the app update to one or more other devices to modify similar operations performed thereon.

According to various example embodiments, one or more of the methodologies described herein may facilitate image-based analysis of a test kit, such as an LFA test kit. Moreover, one or more of the methodologies described herein may facilitate machine-recognition (e.g., via computer vision, artificial intelligence, or both) of one or more test results appearing in an image of a test kit. Hence, one or more of the methodologies described herein may facilitate automated reading of one or more results visible on a test kit, as well as automated instruction of a user in using a test kit (e.g., alone) and obtaining a reliable reading of one or more results thereof, compared to capabilities of pre-existing systems and methods.

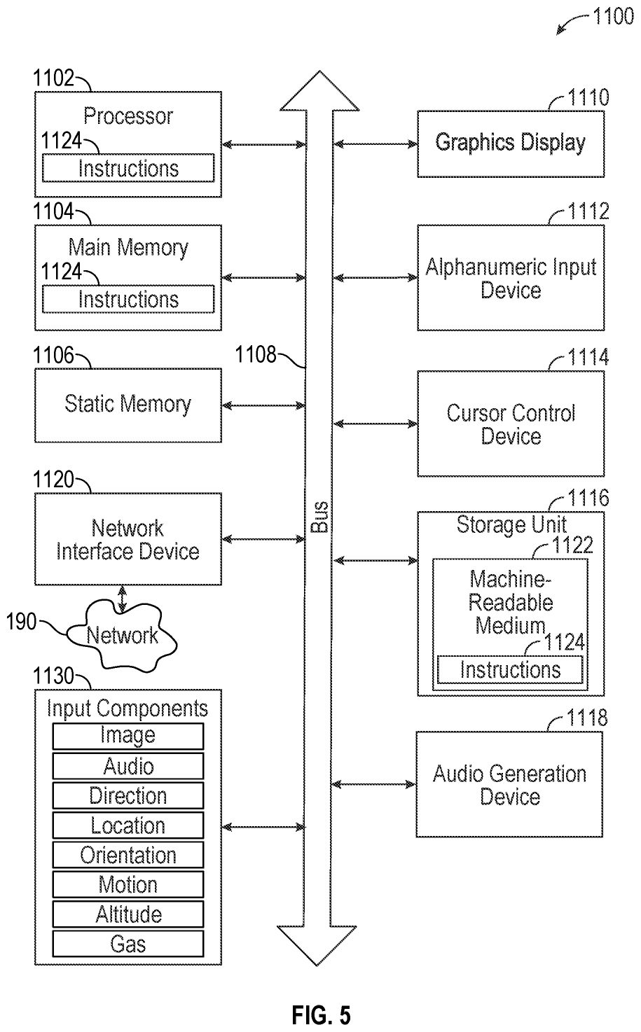

FIG. 5 is a block diagram illustrating components of a machine 1100, according to some example embodiments, able to read instructions 1124 from a machine-readable medium 1122 (e.g., a non-transitory machine-readable medium, a machine-readable storage medium, a computer-readable storage medium, or any suitable combination thereof) and perform any one or more of the methodologies discussed herein, in whole or in part. Specifically, FIG. 5 shows the machine 1100 in the example form of a computer system (e.g., a computer) within which the instructions 1124 (e.g., software, a program, an application, an applet, an app, or other executable code) for causing the machine 1100 to perform any one or more of the methodologies discussed herein may be executed, in whole or in part.

In alternative embodiments, the machine 1100 operates as a standalone device or may be communicatively coupled (e.g., networked) to other machines. In a networked deployment, the machine 1100 may operate in the capacity of a server machine or a client machine in a server-client network environment, or as a peer machine in a distributed (e.g., peer-to-peer) network environment. The machine 1100 may be a server computer, a client computer, a personal computer (PC), a tablet computer, a laptop computer, a netbook, a cellular telephone, a smart phone, a set-top box (STB), a personal digital assistant (PDA), a web appliance, a network router, a network switch, a network bridge, or any machine capable of executing the instructions 1124, sequentially or otherwise, that specify actions to be taken by that machine. Further, while only a single machine is illustrated, the term “machine” shall also be taken to include any collection of machines that individually or jointly execute the instructions 1124 to perform all or part of any one or more of the methodologies discussed herein.

The machine 1100 includes a processor 1102 (e.g., one or more central processing units (CPUs), one or more graphics processing units (GPUs), one or more digital signal processors (DSPs), one or more application specific integrated circuits (ASICs), one or more radio-frequency integrated circuits (RFICs), or any suitable combination thereof), a main memory 1104, and a static memory 1106, which are configured to communicate with each other via a bus 1108. The processor 1102 contains solid-state digital microcircuits (e.g., electronic, optical, or both) that are configurable, temporarily or permanently, by some or all of the instructions 1124 such that the processor 1102 is configurable to perform any one or more of the methodologies described herein, in whole or in part. For example, a set of one or more microcircuits of the processor 1102 may be configurable to execute one or more modules (e.g., software modules) described herein. In some example embodiments, the processor 1102 is a multicore CPU (e.g., a dual-core CPU, a quad-core CPU, an 8-core CPU, or a 128-core CPU) within which each of multiple cores behaves as a separate processor that is able to perform any one or more of the methodologies discussed herein, in whole or in part. Although the beneficial effects described herein may be provided by the machine 1100 with at least the processor 1102, these same beneficial effects may be provided by a different kind of machine that contains no processors (e.g., a purely mechanical system, a purely hydraulic system, or a hybrid mechanical-hydraulic system), if such a processor-less machine is configured to perform one or more of the methodologies described herein.

The machine 1100 may further include a graphics display 1110 (e.g., a plasma display panel (PDP), a light emitting diode (LED) display, a liquid crystal display (LCD), a projector, a cathode ray tube (CRT), or any other display capable of displaying graphics or video). The machine 1100 may also include an alphanumeric input device 1112 (e.g., a keyboard or keypad), a pointer input device 1114 (e.g., a mouse, a touchpad, a touchscreen, a trackball, a joystick, a stylus, a motion sensor, an eye tracking device, a data glove, or other pointing instrument), a data storage 1116, an audio generation device 1118 (e.g., a sound card, an amplifier, a speaker, a headphone jack, or any suitable combination thereof), and a network interface device 1120.

The data storage 1116 (e.g., a data storage device) includes the machine-readable medium 1122 (e.g., a tangible and non-transitory machine-readable storage medium) on which are stored the instructions 1124 embodying any one or more of the methodologies or functions described herein. The instructions 1124 may also reside, completely or at least partially, within the main memory 1104, within the static memory 1106, within the processor 1102 (e.g., within the processor's cache memory), or any suitable combination thereof, before or during execution thereof by the machine 1100. Accordingly, the main memory 1104, the static memory 1106, and the processor 1102 may be considered machine-readable media (e.g., tangible and non-transitory machine-readable media). The instructions 1124 may be transmitted or received over the network 190 via the network interface device 1120. For example, the network interface device 1120 may communicate the instructions 1124 using any one or more transfer protocols (e.g., hypertext transfer protocol (HTTP)).

In some example embodiments, the machine 1100 may be a portable computing device (e.g., a smart phone, a tablet computer, or a wearable device) and may have one or more additional input components 1130 (e.g., sensors or gauges). Examples of such input components 1130 include an image input component (e.g., one or more cameras), an audio input component (e.g., one or more microphones), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), a temperature input component (e.g., a thermometer), and a gas detection component (e.g., a gas sensor). Input data gathered by any one or more of these input components 1130 may be accessible and available for use by any of the modules described herein (e.g., with suitable privacy notifications and protections, such as opt-in consent or opt-out consent, implemented in accordance with user preference, applicable regulations, or any suitable combination thereof).

As used herein, the term “memory” refers to a machine-readable medium able to store data temporarily or permanently and may be taken to include, but not be limited to, random-access memory (RAM), read-only memory (ROM), buffer memory, flash memory, and cache memory. While the machine-readable medium 1122 is shown in an example embodiment to be a single medium, the term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of carrying (e.g., storing or communicating) the instructions 1124 for execution by the machine 1100, such that the instructions 1124, when executed by one or more processors of the machine 1100 (e.g., processor 1102), cause the machine 1100 to perform any one or more of the methodologies described herein, in whole or in part. Accordingly, a “machine-readable medium” refers to a single storage apparatus or device, as well as cloud-based storage systems or storage networks that include multiple storage apparatus or devices. The term “machine-readable medium” shall accordingly be taken to include, but not be limited to, one or more tangible and non-transitory data repositories (e.g., data volumes) in the example form of a solid-state memory chip, an optical disc, a magnetic disc, or any suitable combination thereof.

A “non-transitory” machine-readable medium, as used herein, specifically excludes propagating signals per se. According to various example embodiments, the instructions 1124 for execution by the machine 1100 can be communicated via a carrier medium (e.g., a machine-readable carrier medium). Examples of such a carrier medium include a non-transient carrier medium (e.g., a non-transitory machine-readable storage medium, such as a solid-state memory that is physically movable from one place to another place) and a transient carrier medium (e.g., a carrier wave or other propagating signal that communicates the instructions 1124).

Certain example embodiments are described herein as including modules. Modules may constitute software modules (e.g., code stored or otherwise embodied in a machine-readable medium or in a transmission medium), hardware modules, or any suitable combination thereof. A “hardware module” is a tangible (e.g., non-transitory) physical component (e.g., a set of one or more processors) capable of performing certain operations and may be configured or arranged in a certain physical manner. In various example embodiments, one or more computer systems or one or more hardware modules thereof may be configured by software (e.g., an application or portion thereof) as a hardware module that operates to perform operations described herein for that module.

In some example embodiments, a hardware module may be implemented mechanically, electronically, hydraulically, or any suitable combination thereof. For example, a hardware module may include dedicated circuitry or logic that is permanently configured to perform certain operations. A hardware module may be or include a special-purpose processor, such as a field programmable gate array (FPGA) or an ASIC. A hardware module may also include programmable logic or circuitry that is temporarily configured by software to perform certain operations. As an example, a hardware module may include software encompassed within a CPU or other programmable processor. It will be appreciated that the decision to implement a hardware module mechanically, hydraulically, in dedicated and permanently configured circuitry, or in temporarily configured circuitry (e.g., configured by software) may be driven by cost and time considerations.

Accordingly, the phrase “hardware module” should be understood to encompass a tangible entity that may be physically constructed, permanently configured (e.g., hardwired), or temporarily configured (e.g., programmed) to operate in a certain manner or to perform certain operations described herein. Furthermore, as used herein, the phrase “hardware-implemented module” refers to a hardware module. Considering example embodiments in which hardware modules are temporarily configured (e.g., programmed), each of the hardware modules need not be configured or instantiated at any one instance in time. For example, where a hardware module includes a CPU configured by software to become a special-purpose processor, the CPU may be configured as respectively different special-purpose processors (e.g., each included in a different hardware module) at different times. Software (e.g., a software module) may accordingly configure one or more processors, for example, to become or otherwise constitute a particular hardware module at one instance of time and to become or otherwise constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory (e.g., a memory device) to which it is communicatively coupled. A further hardware module may then, at a later time, access the memory to retrieve and process the stored output. Hardware modules may also initiate communications with input or output devices, and can operate on a resource (e.g., a collection of information from a computing resource).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module in which the hardware includes one or more processors. Accordingly, the operations described herein may be at least partially processor-implemented, hardware-implemented, or both, since a processor is an example of hardware, and at least some operations within any one or more of the methods discussed herein may be performed by one or more processor-implemented modules, hardware-implemented modules, or any suitable combination thereof.

Moreover, such one or more processors may perform operations in a “cloud computing” environment or as a service (e.g., within a “software as a service” (SaaS) implementation). For example, at least some operations within any one or more of the methods discussed herein may be performed by a group of computers (e.g., as examples of machines that include processors), with these operations being accessible via a network (e.g., the Internet) and via one or more appropriate interfaces (e.g., an application program interface (API)). The performance of certain operations may be distributed among the one or more processors, whether residing only within a single machine or deployed across a number of machines. In some example embodiments, the one or more processors or hardware modules (e.g., processor-implemented modules) may be located in a single geographic location (e.g., within a home environment, an office environment, or a server farm). In other example embodiments, the one or more processors or hardware modules may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and their functionality presented as separate components and functions in example configurations may be implemented as a combined structure or component with combined functions. Similarly, structures and functionality presented as a single component may be implemented as separate components and functions. These and other variations, modifications, additions, and improvements fall within the scope of the subject matter herein.

Some portions of the subject matter discussed herein may be presented in terms of algorithms or symbolic representations of operations on data stored as bits or binary digital signals within a memory (e.g., a computer memory or other machine memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “accessing,” “processing,” “detecting,” “computing,” “calculating,” “determining,” “generating,” “presenting,” “displaying,” or the like refer to actions or processes performable by a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The following enumerated descriptions describe various examples of methods, machine-readable media, and systems (e.g., machines, devices, or other apparatus) discussed herein. It should be noted that one or more features of an example, taken in isolation or combination, should be considered within the disclosure of this application.

A first example provides a method comprising:
causing, by one or more processors of a machine, presentation of an instruction that a user of the machine capture an image to depict a test kit used by the user;
detecting, by the one or more processors of the machine, that the image depicting the test kit was captured after expiration of a predetermined minimum wait time and before expiration of a predetermined maximum wait time;
generating, by the one or more processors of the machine, a set of one or more results based on a computer analysis (e.g., computer vision analysis) of the image depicting the test kit and captured after expiration of the predetermined minimum wait time and before expiration of the predetermined maximum wait time; and
providing, by the one or more processors of the machine, at least a result from among the generated set of one or more results based on the computer analysis of the image that depicts the test kit.

A second example provides a method according to the first example, further comprising:
detecting that the user is attempting to capture the image of the test kit prior to expiration of the predetermined minimum wait time; and
causing presentation of a warning that the user is attempting to capture the image of the test kit prior to expiration of the predetermined minimum wait time.

A third example provides a method according to the first example of the second example, further comprising:
detecting that the user has not yet captured the image of the test kit, that the predetermined minimum wait time has expired, and that the predetermined maximum wait time has not expired; and
causing presentation of a prompt that the user capture the image of the test kit.

A fourth example provides a method according to any of the first through third examples, further comprising:
detecting that the user has not yet captured the image of the test kit and that the predetermined maximum wait time is within a predetermined threshold warning period; and
causing presentation of a warning that the user has not yet captured the image of the test kit and that the predetermined maximum wait time is within a predetermined threshold warning period.

A fifth example provides a method according to any of the first through fourth examples, wherein:
the generating of the set of one or more results based on the computer analysis of the image is further based on a computer recognition (e.g., a computer vision recognition) of a results window of the test kit, the results window being depicted in the image of the test kit.

A sixth example provides a method according to any of the first through fifth examples, further comprising:
causing presentation of a graphical user interface operable by the user to define a bounding box around a results window of the test kit depicted in the captured image; and wherein:
the generating of the set of one or more results based on the computer analysis of the image is further based on a computer recognition (e.g., a computer vision recognition) of the results window around which the bounding box is defined by the user.

A seventh example provides a method according to any of the first through sixth examples, further comprising:
generating a prediction of a health status of the user of the machine, the prediction of the health status being generated based on at least one of the set of one or more results generated based on the computer analysis of the image of the test kit.

An eighth example provides a machine-readable medium (e.g., a non-transitory machine-readable storage medium) storing instructions that, when executed by one or more processors of a machine, cause the machine to perform operations comprising:
causing presentation of an instruction that a user of the machine capture an image to depict a test kit used by the user;
detecting that the image depicting the test kit was captured after expiration of a predetermined minimum wait time and before expiration of a predetermined maximum wait time;
generating a set of one or more results based on a computer analysis (e.g., computer vision analysis) of the image depicting the test kit and captured after expiration of the predetermined minimum wait time and before expiration of the predetermined maximum wait time; and
providing at least a result from among the generated set of one or more results based on the computer analysis of the image that depicts the test kit.

A ninth example provides a machine-readable medium according to the eighth example, wherein the operations further comprise:
detecting that the user is attempting to capture the image of the test kit prior to expiration of the predetermined minimum wait time; and
causing presentation of a warning that the user is attempting to capture the image of the test kit prior to expiration of the predetermined minimum wait time.

A tenth example provides a machine-readable medium according to the eighth example or the ninth example, wherein the operations further comprise:
detecting that the user has not yet captured the image of the test kit, that the predetermined minimum wait time has expired, and that the predetermined maximum wait time has not expired; and
causing presentation of a prompt that the user capture the image of the test kit.

An eleventh example provides a machine-readable medium according to any of the eighth through tenth examples, wherein the operations further comprise:
detecting that the user has not yet captured the image of the test kit and that the predetermined maximum wait time is within a predetermined threshold warning period; and
causing presentation of a warning that the user has not yet captured the image of the test kit and that the predetermined maximum wait time is within a predetermined threshold warning period.

A twelfth example provides a machine-readable medium according to any of the eighth through eleventh examples, wherein:
the generating of the set of one or more results based on the computer analysis of the image is further based on a computer recognition (e.g., a computer vision recognition) of a results window of the test kit, the results window being depicted in the image of the test kit.

A thirteenth example provides a machine-readable medium according to any of the eighth through twelfth examples, wherein the operations further comprise:
causing presentation of a graphical user interface operable by the user to define a bounding box around a results window of the test kit depicted in the captured image; and wherein:
the generating of the set of one or more results based on the computer analysis of the image is further based on a computer recognition (e.g., a computer vision recognition) of the results window around which the bounding box is defined by the user.

A fourteenth example provides a machine-readable medium accordingly to any of the eighth through thirteenth examples, wherein the operations further comprise:
generating a prediction of a health status of the user of the machine, the prediction of the health status being generated based on at least one of the set of one or more results generated based on the computer analysis of the image of the test kit.

A fifteenth example provides a system (e.g., a computer system) comprising:
one or more processors; and
a memory storing instructions that, when executed by the one or more processors, cause the system to perform operations comprising:
causing presentation of an instruction that a user (e.g., of the system) capture an image to depict a test kit used by the user;
detecting that the image depicting the test kit was captured after expiration of a predetermined minimum wait time and before expiration of a predetermined maximum wait time;
generating a set of one or more results based on a computer analysis (e.g., computer vision analysis) of the image depicting the test kit and captured after expiration of the predetermined minimum wait time and before expiration of the predetermined maximum wait time; and
providing at least a result from among the generated set of one or more results based on the computer analysis of the image that depicts the test kit.

A sixteenth example provides a system according to the fifteenth example, wherein the operations further comprise:
detecting that the user is attempting to capture the image of the test kit prior to expiration of the predetermined minimum wait time; and
causing presentation of a warning that the user is attempting to capture the image of the test kit prior to expiration of the predetermined minimum wait time.

A seventeenth example provides a system according to the fifteenth example or the sixteenth example, wherein the operations further comprise:
detecting that the user has not yet captured the image of the test kit, that the predetermined minimum wait time has expired, and that the predetermined maximum wait time has not expired; and
causing presentation of a prompt that the user capture the image of the test kit.

An eighteenth example provides a system according to any of the fifteenth through seventeenth examples, wherein the operations further comprise:
detecting that the user has not yet captured the image of the test kit and that the predetermined maximum wait time is within a predetermined threshold warning period; and
causing presentation of a warning that the user has not yet captured the image of the test kit and that the predetermined maximum wait time is within a predetermined threshold warning period.

A nineteenth example provides a system according to any of the fifteenth through eighteenth examples, wherein:
the generating of the set of one or more results based on the computer analysis of the image is further based on a computer recognition (e.g., a computer vision recognition) of a results window of the test kit, the results window being depicted in the image of the test kit.

A twentieth example provides a system according to any of the fifteenth through nineteenth examples, wherein the operations further comprise:
causing presentation of a graphical user interface operable by the user to define a bounding box around a results window of the test kit depicted in the captured image; and wherein:
the generating of the set of one or more results based on the computer analysis of the image is further based on a computer recognition (e.g., a computer vision recognition) of the results window around which the bounding box is defined by the user.

A twenty-first example provides a system according to any of the fifteenth through twentieth examples, wherein:
the generating of the set of one or more results based on the computer analysis of the image is further based on a computer recognition (e.g., a computer vision recognition) of a marking and a corresponding indicator that together indicate a presence of a control within the test kit, the marking and the corresponding indicator being depicted in the image of the test kit.

A twenty-second example provides a carrier medium carrying machine-readable instructions for controlling a machine to carry out the operations (e.g., method operations) performed in any one of the previously described examples.

What is claimed is:

1. A method comprising:
causing, by one or more processors of a machine, presentation of an instruction that a user of the machine capture an image to depict a test kit used by the user;
detecting, by the one or more processors of the machine, that the image depicting the test kit was captured after expiration of a predetermined minimum wait time and before expiration of a predetermined maximum wait time;
generating, by the one or more processors of the machine, a set of one or more results based on a computer analysis of the image depicting the test kit and captured after expiration of the predetermined minimum wait time and before expiration of the predetermined maximum wait time; and
providing, by the one or more processors of the machine, at least a result from among the generated set of one or more results based on the computer analysis of the image that depicts the test kit.

2. The method of claim 1, further comprising:
detecting that the user is attempting to capture the image of the test kit prior to expiration of the predetermined minimum wait time; and
causing presentation of a warning that the user is attempting to capture the image of the test kit prior to expiration of the predetermined minimum wait time.

3. The method of claim 1, further comprising:
detecting that the user has not yet captured the image of the test kit, that the predetermined minimum wait time has expired, and that the predetermined maximum wait time has not expired; and
causing presentation of a prompt that the user capture the image of the test kit.

4. The method of claim 1, further comprising:
detecting that the user has not yet captured the image of the test kit and that the predetermined maximum wait time is within a predetermined threshold warning period; and
causing presentation of a warning that the user has not yet captured the image of the test kit and that the predetermined maximum wait time is within a predetermined threshold warning period.

5. The method of claim 1, wherein:
the generating of the set of one or more results based on the computer analysis of the image is further based on a computer recognition of a results window of the test kit, the results window being depicted in the image of the test kit.

6. The method of claim 1, further comprising:
causing presentation of a graphical user interface operable by the user to define a bounding box around a results window of the test kit depicted in the captured image; and wherein:
the generating of the set of one or more results based on the computer analysis of the image is further based on a computer recognition of the results window around which the bounding box is defined by the user.

7. The method of claim 1, further comprising:
generating a prediction of a health status of the user of the machine, the prediction of the health status being generated based on at least one of the set of one or more results generated based on the computer analysis of the image of the test kit.

8. A machine-readable medium storing instructions that, when executed by one or more processors of a machine, cause the machine to perform operations comprising:
causing presentation of an instruction that a user of the machine capture an image to depict a test kit used by the user;
detecting that the image depicting the test kit was captured after expiration of a predetermined minimum wait time and before expiration of a predetermined maximum wait time;
generating a set of one or more results based on a computer analysis of the image depicting the test kit and captured after expiration of the predetermined minimum wait time and before expiration of the predetermined maximum wait time; and
providing at least a result from among the generated set of one or more results based on the computer analysis of the image that depicts the test kit.

9. The machine-readable medium of claim 8, wherein the operations further comprise:
detecting that the user is attempting to capture the image of the test kit prior to expiration of the predetermined minimum wait time; and
causing presentation of a warning that the user is attempting to capture the image of the test kit prior to expiration of the predetermined minimum wait time.

10. The machine-readable medium of claim 8, wherein the operations further comprise:
detecting that the user has not yet captured the image of the test kit, that the predetermined minimum wait time has expired, and that the predetermined maximum wait time has not expired; and
causing presentation of a prompt that the user capture the image of the test kit.

11. The machine-readable medium of claim 8, wherein the operations further comprise:
detecting that the user has not yet captured the image of the test kit and that the predetermined maximum wait time is within a predetermined threshold warning period; and
causing presentation of a warning that the user has not yet captured the image of the test kit and that the predetermined maximum wait time is within a predetermined threshold warning period.

12. The machine-readable medium of claim 8, wherein:
the generating of the set of one or more results based on the computer analysis of the image is further based on a computer recognition of a results window of the test kit, the results window being depicted in the image of the test kit.

13. The machine-readable medium of claim 8, wherein the operations further comprise:
causing presentation of a graphical user interface operable by the user to define a bounding box around a results window of the test kit depicted in the captured image; and wherein:
the generating of the set of one or more results based on the computer analysis of the image is further based on a computer recognition of the results window around which the bounding box is defined by the user.

14. The machine-readable medium of claim 8, wherein the operations further comprise:
generating a prediction of a health status of the user of the machine, the prediction of the health status being generated based on at least one of the set of one or more results generated based on the computer analysis of the image of the test kit.

15. A system comprising:
one or more processors; and
a memory storing instructions that, when executed by the one or more processors, cause the system to perform operations comprising:
causing presentation of an instruction that a user capture an image to depict a test kit used by the user;
detecting that the image depicting the test kit was captured after expiration of a predetermined minimum wait time and before expiration of a predetermined maximum wait time;
generating a set of one or more results based on a computer analysis of the image depicting the test kit and captured after expiration of the predetermined minimum wait time and before expiration of the predetermined maximum wait time; and
providing at least a result from among the generated set of one or more results based on the computer analysis of the image that depicts the test kit.

16. The system of claim 15, wherein the operations further comprise:
detecting that the user is attempting to capture the image of the test kit prior to expiration of the predetermined minimum wait time; and
causing presentation of a warning that the user is attempting to capture the image of the test kit prior to expiration of the predetermined minimum wait time.

17. The system of claim 15, wherein the operations further comprise:
detecting that the user has not yet captured the image of the test kit, that the predetermined minimum wait time has expired, and that the predetermined maximum wait time has not expired; and
causing presentation of a prompt that the user capture the image of the test kit.

18. The system of claim 15, wherein the operations further comprise:
detecting that the user has not yet captured the image of the test kit and that the predetermined maximum wait time is within a predetermined threshold warning period; and
causing presentation of a warning that the user has not yet captured the image of the test kit and that the predetermined maximum wait time is within a predetermined threshold warning period.

19. The system of claim 15, wherein:
the generating of the set of one or more results based on the computer analysis of the image is further based on a computer recognition of a results window of the test kit, the results window being depicted in the image of the test kit.

20. The system of claim 15, wherein the operations further comprise:
causing presentation of a graphical user interface operable by the user to define a bounding box around a results window of the test kit depicted in the captured image; and wherein:
the generating of the set of one or more results based on the computer analysis of the image is further based on a computer recognition of the results window around which the bounding box is defined by the user.

21. The system of claim 15, wherein:
the generating of the set of one or more results based on the computer analysis of the image is further based on a computer recognition of a marking and a corresponding indicator that together indicate a presence of a control within the test kit, the marking and the corresponding indicator being depicted in the image of the test kit.
